(19) United States
(12) Patent Application Publication
(10) Pub. No.: US 2003/0050627 A1
(43) Pub. Date: Mar. 13, 2003

(54) Adjustable depth lancing device

(76) Inventors: William C. Taylor, Alpharetta, GA (US); Gwenn E. Kennedy, Ellenwood, GA (US)

Correspondence Address: GARDNER GROFF & MEHRMAN, P.C., PAPER MILL VILLAGE, BUILDING 23, 600 VILLAGE TRACE, SUITE 300, MARIETTA, GA, 30067, US

(21) Appl. No.: 10/242,912

(22) Filed: Sep. 13, 2002

Related U.S. Application Data
(60) Provisional application No. 60/318,959, filed on Sep. 13, 2001.
(60) Provisional application No. 60/344,469, filed on Nov. 9, 2001.

(51) Int. Cl. A61B 17/00

(52) U.S. Cl. 606/1; 604/411

(57) ABSTRACT

A lancing housing has an endcap with an opening, and a lancing element has a needle that extends through the opening and beyond the endcap an adjustable distance selected to provide the desired depth of needle penetration. In exemplary embodiments, the distance the needle extends from the endcap is adjusted by a removable raised ring on the endcap, projections and cooperating recesses for positioning the endcap spaced apart from the housing, multi-positionable lancing element stops on the inside of the endcap, or a flip-out spacer adjacent the endcap. In preferred form, a disabling mechanism prevents re-arming the device after a single use. Additionally, a method of lancing the skin of a subject is provided for use with any adjustable penetration depth lancing device.

### CROSS-REFERENCE TO RELATED APPLICATIONS

This application claims the priority benefit of U.S. Provisional Patent Application Serial No. 60/318,959, filed Sep. 13, 2001, and U.S. Provisional Patent Application Serial No. 60/344,469, filed Nov. 9, 2001, both of which are hereby incorporated herein by reference in their entirety.

### FIELD OF THE INVENTION

The present invention relates generally to medical devices and, more particularly, to a lancing device for penetrating the skin of a human or animal subject for sampling of blood and/or other body fluids.

### BACKGROUND OF THE INVENTION

Lancing devices are used to penetrate the skin of a subject and obtain a sample of blood or other body fluid, as in the testing of blood sugar levels by diabetics. Typically, a lancet having a sharp point is translationally mounted within a housing portion of a lancing device. The lancet is driven by a spring or other biasing means to cause the sharp point to extend a small distance through an opening in the housing and into the subject's skin, creating a wound from which the sample of body fluid is collected. The housing optionally includes a pressure surface for “pumping” the wound to enhance sample size, and may also incorporate a capillary tube or other sample collection media. The endcap of the housing or a portion of the housing adjacent the lancet opening may include an open window or a transparent section for viewing the sample collection site, and may also include one or more sample size indicators for comparing the size of a sample to a desired sample size. Example lancing devices are shown in U.S. Pat. No. 5,356,420; U.S. Pat. No. 5,397,334; and U.S. Pat. No. 5,439,473, all of which are hereby incorporated herein by reference.

Lancing devices typically are intended either for a single use or for multiple uses. Single-use lancing devices generally are disposed of after one use. For example, in a hospital or clinic, it is desirable to provide a single-use lancing device that can be used on a patient and then disposed of to eliminate any risk of infection to subsequent patients or caregivers from exposure to residual body fluids remaining on the lancing device. Accordingly, single-use lancing devices oftentimes include a disabling mechanism to prevent accidental or intentional re-use of the device. Various forms of disabling mechanisms are available, and are well known in the art. For example, the disabling mechanism may comprise a return spring for retracting the sharp point of the lancet back into the housing after a single use, break-away elements or a frangible link in the cocking or triggering mechanism to prevent rearming or re-firing the device after a single use, a locking element, and/or a shield for blocking travel of the lancet.

Because single-use lancing devices normally are disposed of after one use, they generally are relatively simple in construction so that they can be economically manufactured in large quantities. To keep the design simple and economical, known single-use lancing devices do not include adjustability features. For example, known single-use lancing devices typically do not provide for adjustment of the depth of penetration of the lancet needle beneath the surface of the subject's skin. Accordingly, users of single-use lancing devices have little or no ability to adjust the depth and/or size of the wound in order to control the fluid sample size. While one user may be able to obtain a sufficient sample from a relatively small wound, another user who bleeds less freely may require a larger wound size to generate a sample of the same size. As a result, a user may be forced to suffer the pain of a lancet stick that is deeper than necessary to collect a sample of adequate size, or may need more than one lancet stick to generate a sufficient sample size. The lack of depth adjustment also renders previously known single-use lancing devices generally unsuitable for use in sampling from different body sites. For a given user, obtaining a sample of a specified size from a forearm sampling site typically requires a greater depth of penetration than obtaining the same size sample from a fingertip sampling site. Thus, previously known single-use sampling devices render it difficult or impossible for a user to obtain just the proper sample size from a single lancet stick, or to sample from different sites on the body.

Accordingly, a need exists for lancing devices providing depth adjustment of the lancet needle. In addition, it would be desirable for such lancing devices to be simple and economical in design, manufacture, and use so that they could be incorporated into single-use lancing devices. Furthermore, such lancing devices are needed that permit individual users to obtain the proper size and depth of wound for drawing fluid, without unnecessarily and repeatedly sticking oneself, without enduring the pain of deeper than needed sticks, and without the risk of contamination from any previous users. It is to the provision of single-use lancing devices meeting these and other needs that the present invention is primarily directed.

### SUMMARY OF THE INVENTION

The present invention is a lancing device providing selective adjustment of the depth of penetration of the lancet point. The lancing device is preferably a single-use lancing device, but alternatively can be a multi-use lancing device. The lancing device comprises a housing having an endcap with an opening, and a lancing element having a needle with a point or a sharp edge. The needle point is movable between a retracted position and extended positions, the extended positions spaced an adjustable distance from the endcap to provide the desired depth of needle penetration. A distance adjusting feature permits extending the needle point to a selected one of the multiple extended positions.

Preferably, the lancing device includes a trigger mechanism operable to “fire” the lancing device by propelling the needle point from the retracted position to the extended positions. Additionally, the lancing device may include an indicator means for showing the distance adjustment setting. In single-use forms, the lancing device also has a disabling mechanism for preventing re-use of the lancing device after a single use.

In a first exemplary embodiment, the distance adjusting feature is provided by a raised ring surrounding at least a portion of the opening. The raised ring is detachable from the remainder of the housing to permit exposing a greater length of the lancing element needle. Additionally, the raised ring may include a pull tab for grasping to remove the ring from the housing. In operation, the needle point extends a first distance from the housing raised ring when the ring is attached to the housing and the needle point extends a second distance from the housing endcap when the ring is removed from the housing.

In a second exemplary embodiment, the distance adjusting feature is provided by one or more projections on the endcap that are received in one or more recesses in the housing. Alternatively, the projections may be provided on the housing and the recesses on the endcap. In any event, the cooperating recesses and projections permit positioning the endcap on the body in different positions to expose different lengths of the lancing element needle. For example, two recesses with different lengths can be provided in the body while one projection is provided on the endcap, so that the endcap is spaced apart from the body when in the second position. In operation, the needle point extends a first distance from the endcap when the endcap is coupled to the housing in a first position and the needle point extends a second distance from the endcap when the endcap is coupled to the housing in a second position.

In a third exemplary embodiment, the distance adjusting feature is provided by at least two stops on an inside face of the endcap. The stops selectively contact a cooperating portion of the lancing element to limit lancing element travel. As an example, each of the stops may be provided by a pair of stop arms at opposing sides of the opening. And at least one of the stops extends from the endcap a greater distance than another one of the stops. In operation, the needle point extends a first distance from the endcap when the endcap is rotated so that one of the stops contacts the lancing element and the needle point extends a second distance from the endcap when the endcap is rotated so that another one of the stops contacts the lancing element.

In a fourth exemplary embodiment, the distance adjusting feature is provided by a flip-out spacer pivotally mounted adjacent an inside face of the endcap. The spacer contacts a cooperating portion of the lancing element in a first position of the flip-out spacer to limit lancing element travel and avoids contact with the lancing element in a second position of the flip-out spacer. Preferably, the flip-out spacer has a recess for receiving the cooperating portion of the lancing element to permit the cooperating portion to travel to close to the endcap. And the flip-out spacer has a flip tab for grasping to pivot the spacer out of the housing. In operation, the needle point extends a first distance from the endcap when the flip-out spacer is in the first position and the needle point extends a second distance from the endcap when the flip-out spacer is in the second position.

In addition, the present invention includes a method of lancing the skin of a subject. The method includes the steps of determining a desired lancing penetration depth, adjusting a housing of a lancing device to a setting for a lancing element that corresponds to the desired penetration depth, lancing the skin of the subject with the lancing device, and disabling and disposing of the lancing device after a single use.

Accordingly, the present invention includes a lancing device with a depth adjustment feature for advantageously selecting the depth of penetration of the lancet point. The depth adjustment feature advantageously enables a user to minimize pain and required healing time, while ensuring that the proper sample size can be collected from a sampling site using a single lancet stick. In addition, the depth adjustment feature advantageously enables a user to obtain samples of the desired size from different sampling sites on the body. Furthermore, the depth adjustment feature is provided by structures that are sufficiently simple and economical for use in disposable single-use lancing devices.

These and other features and advantages of the present invention will become more apparent upon reading the following description in conjunction with the accompanying drawing figures.

### BRIEF DESCRIPTION OF THE DRAWING FIGURES

FIG. 1 is a perspective view of a lancing device according to a first exemplary embodiment of the present invention.

FIG. 2 is an end view of the lancing device of FIG. 1.

FIG. 3 is a top view of the lancing device of FIG. 1.

FIG. 4 is a cross-sectional view of the lancing device taken at line 4-4 of FIG. 3, showing the major components thereof.

FIG. 5 is a detail view of the ring of the lancing device of FIG. 4.

FIG. 6 is an end view of the lancing device of FIG. 1, showing the ring removed.

FIG. 7 is a cross-sectional view of the lancing device taken at line 7-7 of FIG. 6, showing the lancing element in the retracted position.

FIG. 8 is an end view of the lancing device of FIG. 6.

FIG. 9 is a cross-sectional view of the lancing device taken at line 9-9 of FIG. 8, showing the lancing element in the first extended position.

FIG. 10 is an end view of the lancing device of FIG. 1, showing the ring intact.

FIG. 11 is a cross-sectional view of the lancing device taken at line 11-11 of FIG. 10, showing the lancing element in the retracted position.

FIG. 12 is an end view of the lancing device of FIG. 10.

FIG. 13 is a cross-sectional view of the lancing device taken at line 13-13 of FIG. 12, showing the lancing element in the second extended position.

FIG. 14 is a perspective view of the lancing device of FIG. 1, showing the ring intact.

FIG. 15 is a perspective view of the lancing device of FIG. 14, showing the initiation of the removal of the ring.

FIG. 16 is a perspective view of the lancing device of FIG. 14, showing the progressive removal of the ring.

FIG. 17 is a perspective view of the lancing device of FIG. 14, showing the removal of the ring almost completed.

FIG. 18 is a perspective view of the lancing device of FIG. 14, showing the ring removed.

FIG. 19 is a perspective view of a lancing device according to a second exemplary embodiment of the present invention.

FIG. 19A is a perspective view of an alternative lancing device according to a second exemplary embodiment of the present invention.

FIG. 20 is an end view of the lancing device of FIG. 19.

FIG. 21 is a top view of the lancing device of FIG. 19.

FIG. 22 is a cross-sectional view of the lancing device taken at line 22-22 of FIG. 21, showing the major components thereof.

FIG. 23 is an end view of the lancing device of FIG. 19, with the endcap in the first position.

FIG. 24 is a cross-sectional view of the lancing device taken at line 24-24 of FIG. 23, showing the lancing element in the retracted position and the endcap in the first position spaced apart from the housing.

FIG. 25 is an end view of the lancing device of FIG. 23.

FIG. 26 is a cross-sectional view of the lancing device taken at line 26-26 of FIG. 25, showing the lancing element in the first extended position.

FIG. 27 is an end view of the lancing device of FIG. 19, with the endcap in the second position.

FIG. 28 is a cross-sectional view of the lancing device taken at line 28-28 of FIG. 27, showing the lancing element in the retracted position and the endcap in the second position adjacent the housing.

FIG. 29 is an end view of the lancing device of FIG. 27.

FIG. 30 is a cross-sectional view of the lancing device taken at line 30-30 of FIG. 29, showing the lancing element in the second extended position.

FIG. 31 is a perspective view of the lancing device of FIG. 19, showing the endcap in the first position spaced apart from the housing.

FIG. 32 is a perspective view of the lancing device of FIG. 31, showing the endcap being rotated from the first position to the second position.

FIG. 33 is a perspective view of the lancing device of FIG. 31, showing the endcap in the second position adjacent the housing.

FIG. 34 is a perspective view of a lancing device according to a third exemplary embodiment of the present invention.

FIG. 35 is a top view of the lancing device of FIG. 34.

FIG. 36 is a cross-sectional view of the lancing device taken at line 36-36 of FIG. 35, showing the major components thereof.

FIG. 37 is a cross-sectional view of the lancing device taken at line 37-37 of FIG. 36.

FIG. 38 is an end view of the lancing device of FIG. 34.

FIG. 39 is an end view of the lancing device of FIG. 34, with the endcap rotated to limit the lancing element travel by the long stops.

FIG. 40 is a cross-sectional view of the lancing device taken at line 40-40 of FIG. 39, showing the lancing element in the retracted position.

FIG. 41 is an end view of the lancing device of FIG. 39.

FIG. 42 is a cross-sectional view of the lancing device taken at line 42-42 of FIG. 41, showing the lancing element stopped in the first extended position by the long stops.

FIG. 43 is an end view of the lancing device of FIG. 34, with the endcap rotated to limit the lancing element travel by the intermediate stops.

FIG. 44 is a cross-sectional view of the lancing device taken at line 44-44 of FIG. 43, showing the lancing element in the retracted position.

FIG. 45 is an end view of the lancing device of FIG. 43.

FIG. 46 is a cross-sectional view of the lancing device taken at line 46-46 of FIG. 45, showing the lancing element stopped in the intermediate extended position by the intermediate stops.

FIG. 47 is an end view of the lancing device of FIG. 34, with the endcap rotated to limit the lancing element travel by the short stops.

FIG. 48 is a cross-sectional view of the lancing device taken at line 48-48 of is FIG. 47, showing the lancing element in the retracted position.

FIG. 49 is an end view of the lancing device of FIG. 47.

FIG. 50 is a cross-sectional view of the lancing device taken at line 50-50 of FIG. 49, showing the lancing element stopped in the second extended position by the short stops.

FIG. 51 is a perspective view of a lancing device according to a fourth exemplary embodiment of the present invention.

FIG. 52 is an end view of the lancing device of FIG. 51.

FIG. 53 is a top view of the lancing device of FIG. 51.

FIG. 54 is a cross-sectional view of the lancing device taken at line 54-54 of FIG. 53, showing the major components thereof.

FIG. 55 is a side view of the ring of the lancing device of FIG. 51.

FIG. 56 is an end view of the lancing device of FIG. 51, showing the spacer in the first position in the housing.

FIG. 57 is a cross-sectional view of the lancing device taken at line 57-57 of FIG. 56, showing the lancing element in the retracted position.

FIG. 58 is an end view of the lancing device of FIG. 56.

FIG. 59 is a cross-sectional view of the lancing device taken at line 59-59 of FIG. 58, showing the lancing element in the first extended position with its travel limited by the spacer.

FIG. 60 is an end view of the lancing device of FIG. 51, showing the spacer in the second position pivoted out of the housing.

FIG. 61 is a cross-sectional view of the lancing device taken at line 61-61 of FIG. 60, showing the lancing element in the retracted position.

FIG. 62 is an end view of the lancing device of FIG. 60.

FIG. 63 is a cross-sectional view of the lancing device taken at line 63-63 of FIG. 62, showing the lancing element in the second extended position with its travel not limited by the spacer.

FIG. 64 is a perspective view of the lancing device of FIG. 51, showing the spacer in the first position in the housing.

FIG. 65 is a perspective view of the lancing device of FIG. 64, showing the initiation of the removal of the spacer from the housing.

FIG. 66 is a perspective view of the lancing device of FIG. 64, showing the progressive pivoting of the spacer out of the housing.

FIG. 67 is a perspective view of the lancing device of FIG. 64, showing the pivoting of the spacer from the housing almost completed.

FIG. 68 is a perspective view of the lancing device of FIG. 64, showing the spacer in the second position pivoted out of the housing.

### DETAILED DESCRIPTION OF EXEMPLARY EMBODIMENTS

Referring now to the drawing figures, in which like reference numbers refer to like parts throughout, exemplary forms of the present invention will now be described by way of example embodiments. It is to be understood that the embodiments described and depicted herein are only selected examples of the many and various forms that the present invention may take, and that these examples are not intended to be exhaustive or limiting of the claimed invention.

Generally described, the present invention provides a lancing device having a housing, a lancing element, and an adjustment mechanism for adjusting the distance that the lancing element will extend from the housing when the lancing device is operated. The housing has an endcap with an opening formed in it. The endcap and housing can be separate parts or a single part. The lancing element has a needle with a point (a tip, blade edge or other piercing element) that travels between a retracted position and extended positions with the needle point extending through the opening and beyond the endcap.

The adjustment mechanism operates to adjust the distance that the point of the lancing element needle extends beyond the endcap of the housing. For example, the lancing device can be adjusted so that the needle point extends a first distance beyond the endcap of the housing or a second distance beyond the endcap of the housing. In some embodiments, the lancing device can be further adjusted so that the needle point extends to another distance beyond the endcap. In a typical commercial embodiment, in one extended position the needle point is about 0.65 mm beyond the endcap and in another extended position the needle point is about 0.4 mm beyond the endcap.

The adjustment mechanism may be provided by a number of different structures, several of which are described in the following exemplary embodiments. These adjustment mechanisms are simple in design and cost-efficient to manufacture, so that lancing devices including them can be made for disposable single-use purposes. But because they are also durable and reliable, these adjustment mechanisms can also be incorporated into multi-use lancing devices.

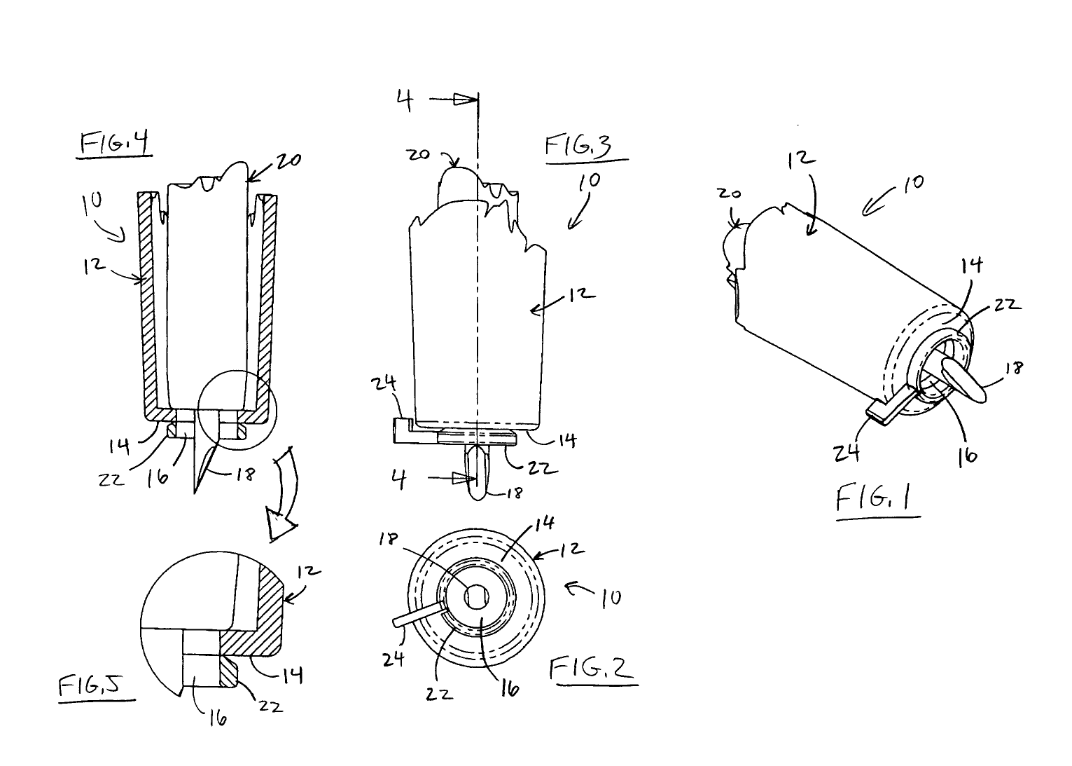

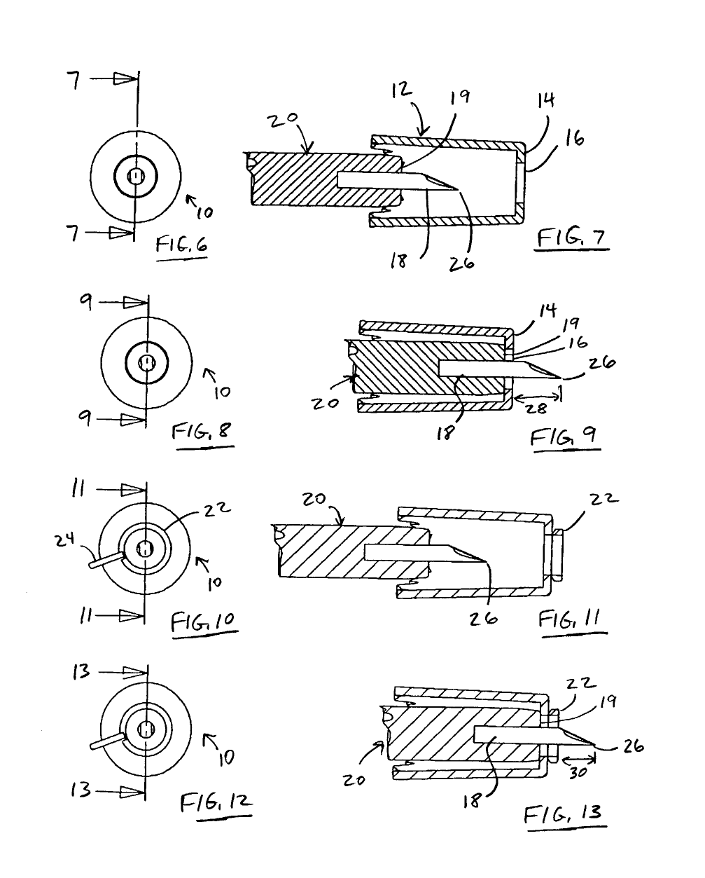

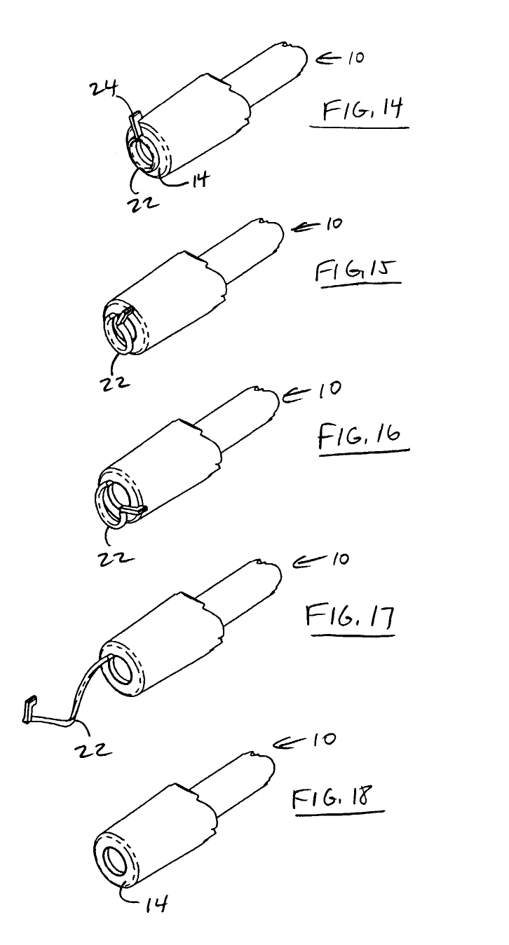

Referring to FIGS. 1-5, there is shown a first exemplary embodiment of the present invention, referred to generally as the lancing device 10. The lancing device 10 includes a housing 12, preferably comprising a generally hollow shell formed of plastic or other material providing sufficient structural rigidity to permit a user to grip and actuate the lancing device to penetrate the skin for sampling. The lancing device 10 further comprises an endcap 14. The endcap 14 may be formed as a separate component from the housing 12 or may be integrally formed with it. The endcap 14 has an opening 16 through its front face for permitting at least a portion of a needle 18 of a lancet or other lancing element 20 to pass therethrough to penetrate the skin of a user for sampling. The opening 16 is preferably sized and/or shaped to permit passage of a portion of the lancing element needle 18, but block the entire lancing element 20 from passing therethrough. This prevents unintended discharge of the lancing element 20 from containment within the housing 12 and endcap 14.

In this embodiment, the adjustment mechanism is provided by a raised ring 22 surrounding at least a portion of the opening 16 through which the lancet needle 18 is driven. The ring 22 can be made of a bead of, for example, soft plastic, rubber, or another elastomeric material. The ring 22 can be integrally formed with the endcap 14 during manufacture of the housing 12, or it can be formed separately and attached to the endcap by, for example, an epoxy or other adherent.

The ring 22 is selectively detachable from the remainder of the endcap 14. For example, the ring 22 may have a portion adjacent the endcap 14 that is sufficiently thin that it fails there and separates from the endcap when a user applies a separating force to the ring. In addition, a pull tab 24 can be attached to the ring to facilitate its removal. In this way, the user can remove the ring 22 to provide deeper penetration of the needle 18 into the skin, or can leave the ring attached to the endcap to provide shallower penetration of the needle.

It will be understood that many alternative forms of this embodiment may be provided. While the housing is shown as cylindrical, it could be rectangular, triangular, polygonal, or otherwise shaped. Similarly, the ring can be circular or another shape conforming to or different from the housing. In addition, the lancing device may be provided with a plurality if rings configured for removing a selected one or more of the rings to produce the desired penetration depth. For example, two rings may be configured so that one ring can be removed while leaving the other one to produce an intermediate penetration depth. Such rings may be provided in a stacked or coaxial arrangement.

FIGS. 6-9 show the operation of the lancing device 10 with the ring removed for providing a deeper lancing penetration. The lancing device is positioned with the forward face of the endcap 14 in contact with the user's skin (not shown) at the desired sampling site. In FIGS. 6 and 7, the lancing element 20 is cocked in the retracted position and ready for use. In FIGS. 8 and 9, the lancing element 20 has been “fired” by an actuating mechanism such that the lancing element travels to a first extended position where at least a portion of the needle 18 passes through the opening 16 and a point 26 of the needle extends beyond the endcap 14 by a first distance 28. A forward face or other cooperating portion 19 of the lancing element 20 impacts the inner face of the endcap 14 or another component of the housing 12 to limit the travel of the lancing element. In the first extended position of the lancing element 20, the needle point 26 penetrates the skin by about the first distance 28 to form a wound for obtaining a sample of blood or other body fluid. With the ring 22 removed, the forward face of the endcap 14 directly contacts the user's skin, permitting the needle to penetrate the skin to a greater depth.

FIGS. 10-13 show the operation of the lancing device 10 with the ring intact for providing a shallower lancing penetration. In FIGS. 10 and 11, the lancing element 20 is cocked in the retracted position and ready for use. In FIGS. 12 and 13, the lancing element 20 has been “fired” by the actuating mechanism such that the lancing element travels to a second extended position where the needle point 26 extends beyond the endcap 14 by a second distance 30. With the ring 22 intact on the endcap 14, it serves as a spacer, adding distance between the inner face of the endcap and the user's skin, thereby reducing the depth of penetration. Accordingly, in this second extended position of the lancing element 20, the needle point 26 penetrates the skin by about the second distance 30 to form a wound for obtaining a sample of blood or other body fluid, with the wound being shallower than that produced with the ring removed. In this way, by selectively removing the ring 22 or leaving the ring in place on the endcap 14, the user can adjust the depth of penetration provided by the lancing device 10.

FIGS. 14-18 show an exemplary method of removing the ring 22 from the endcap 14. In FIG. 14, the ring 22 is intact in the endcap 14. In FIG. 15, the pull tab 24 is being pulled to begin separating the ring 22 from the endcap 14. In FIGS. 16 and 17, the ring 22 is continuing to be separated from the endcap 14. And in FIG. 18, the ring 22 has been completely removed from the endcap 14.

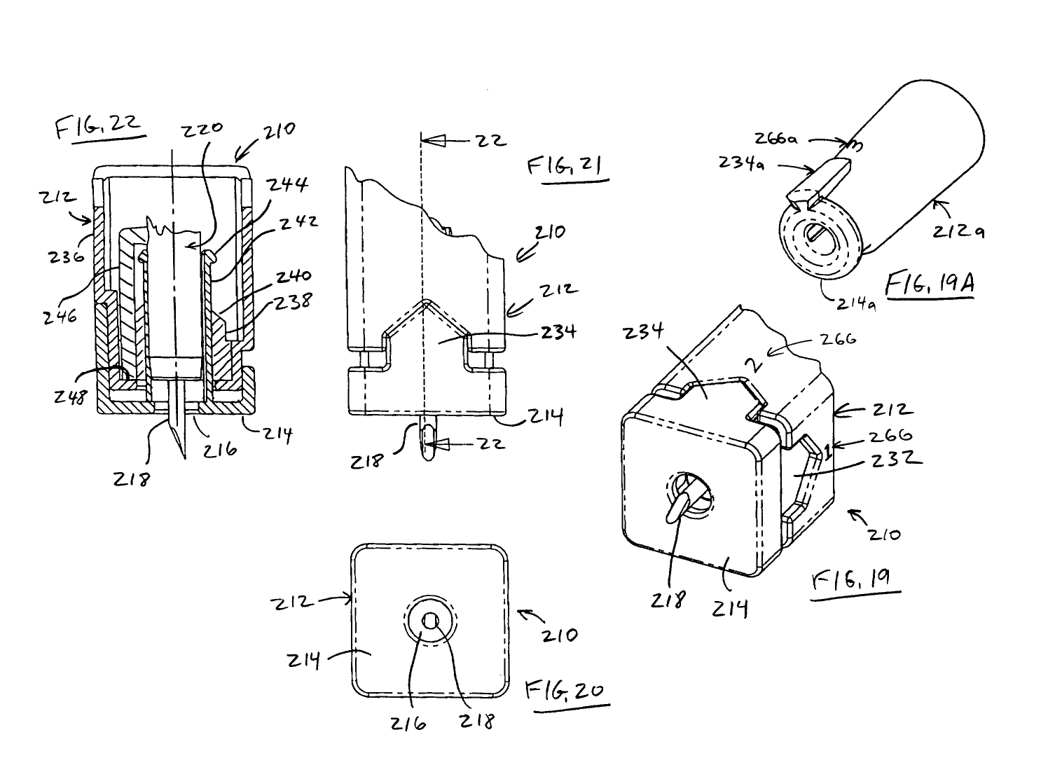

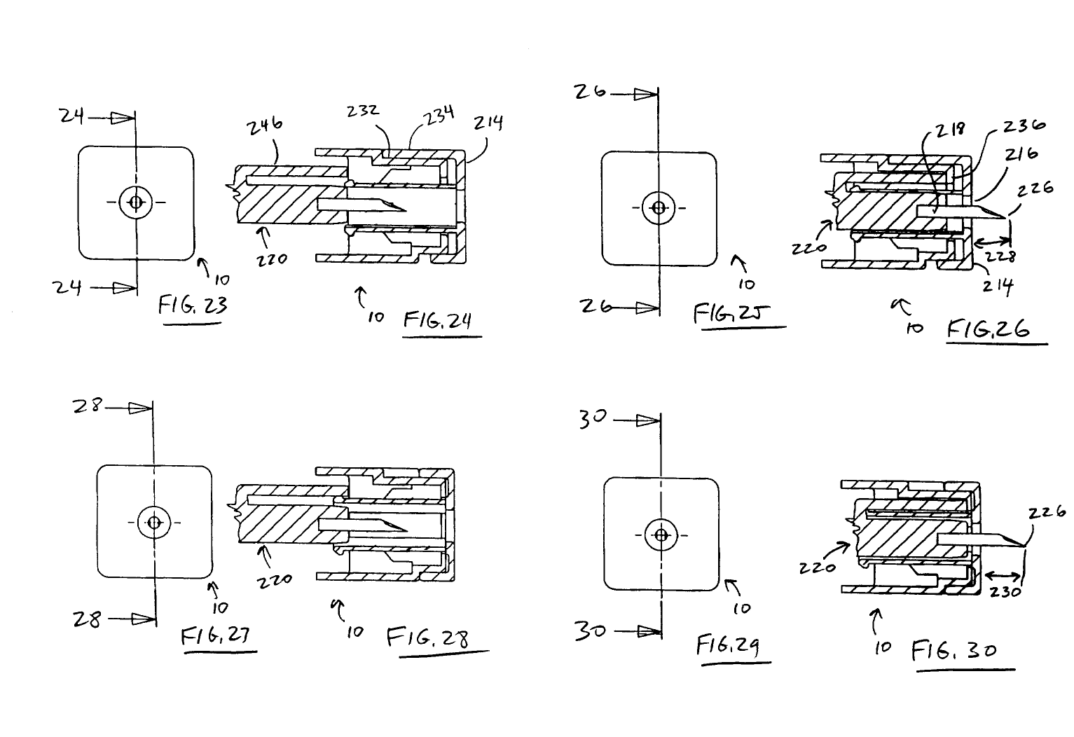

Referring to FIGS. 19-22, there is shown a second exemplary embodiment of the present invention, referred to generally as the lancing device 210. The lancing device 210 includes a housing 212 and an endcap 214 with an opening 216 for permitting at least a portion of a needle 218 of a lancet or other lancing element 220 to pass therethrough to penetrate the skin of a user for sampling. In this embodiment, the adjustment mechanism includes the housing 212 of the lancing device 210 having one or more recesses 232 into which a cooperating projection 234 of the endcap 214 is engaged. The provision of multiple recesses 232 of different depths, and/or at least one recess 232 along with areas of no recess, permit the user to selectively vary the extent to which the projection 234 extends into the housing 212 and thereby vary the effective overall housing length and the corresponding depth of penetration of the lancet needle point into the subject's skin. For example, a deeper one of the recesses 232 accommodates substantially all of the projection 234 and allows the endcap 214 to be further recessed into the housing 212, resulting in a shorter effective housing length, exposing a greater length of the lancing element needle 218, and producing a deeper wound. A shallower recess 232 (or an area of no recess) does not permit the endcap 214 to be pressed as far into or onto the housing 212, resulting in a longer effective housing length because the endcap is spaced apart from the body, exposing a shorter length of the lancing element needle 218, and producing a correspondingly shallower wound.

In addition, as best shown in FIG. 22, the body 236 of the housing 212 has an inner wall 238 with at least one catch surface 240 formed on it, the endcap 214 has at least one guide member 242 extending into the body, and the guide member has a retainer 246 that engages the catch surface. This prevents the endcap 214 from being removed from the body 214. The guide members 242 may be provided in a variety of different configurations. For example, one guide member 242 may be configured as a sleeve extending all the way the lancing element 220, a plurality of guide members may be configured as arms, or the guide members may be otherwise configured.

The inner wall 238 of the housing body 236 also has a stop surface 246 formed on it, and the lancing element 220 has at least one stop member 248 extending toward the inner wall. In this way, the stop member 248 contacts the stop surface 246 when the lancing element 220 is in the extended position to limit lancing element travel. The stop member 248 may be provided in a variety of different configurations. For example, one stop member 248 may be configured as a sleeve extending all the way the lancing element 220 and over the guide member 242, a plurality of stop members may be configured as arms, or the stop members may be otherwise configured.

It will be understood that many alternative forms of this embodiment may be provided. For example, the reverse configuration, wherein the recesses are in the endcap and the projection is on the housing, is also within the scope of the present invention, as well as is a combination arrangement with one or more projections and one or more recesses on each of the housing and the endcap. For example, the endcap may have one recess in it and the housing two projections, with the projections extending longitudinally toward the endcap and being of different length or extending radially outward and being of the same length but one being positioned closer to the endcap than the other.

In addition, the projections and recesses may be provided in any of a variety of shapes, sizes, and numbers, including but not limited to those shown. For example, the lancing device may be provided with one projection and four cooperating recesses of different lengths, with three projections of different lengths, one operating recess for adjusting the penetration depth, and one parking recess deep enough to receive the longest projection when a shorter one is in the operating recess, or with multiple projections and multiple cooperating recesses.

And while the housing and endcap are shown as rectangular, they could be circular (see housing 212a and endcap 214a of FIG. 19A), triangular, polygonal, or otherwise shaped. In this way, projections and/or recesses may be provided in each side to provide the desired adjustability of penetration depth. For example, a triangular shaped housing and endcap, with the housing having a recess in each side, can provide adjustability for three penetration depths. Or an octagonal housing and endcap, with the housing having a recess in each side, can provide adjustability for eight penetration depths.

Furthermore, the endcap is shown capped over and removably coupled to a collar of the housing body, which coupling may be provided by a detent, a snap flange, a tight fit, or another conventional removable coupling mechanism. Alternatively, the recesses may be formed in the inner wall of the housing body, with the endcap projections fitting within the housing body.

FIGS. 23-26 show the operation of the lancing device 210 with the projection 234 in a shallower recess 232 so that the endcap 214 is in a first position for providing a shallower lancing penetration. The lancing device is positioned with the forward face of the endcap 214 in contact with the user's skin (not shown) at the desired sampling site. In FIGS. 23 and 24, the lancing element 220 is cocked in the retracted position and ready for use. In FIGS. 25 and 26, the lancing element 220 has been “fired” by an actuating mechanism such that the lancing element travels to a first extended position where at least a portion of the needle 218 passes through the opening 216 and a point 226 of the needle extends beyond the endcap 214 by a first distance 228. In the first extended position of the lancing element 220, the needle point 226 penetrates the skin by about the first distance 228 to form a wound for obtaining a sample of blood or other body fluid. With the endcap 214 in the first position, the forward face of the endcap 14 is spaced apart from the body of the housing 212, permitting the needle to penetrate the skin only to a shallower depth.

FIGS. 27-30 show the operation of the lancing device 210 with the projection (not shown) in a deeper recess (not shown) so that the endcap 214 is in a second position for providing a deeper lancing penetration. In FIGS. 27 and 28, the lancing element 220 is cocked in the retracted position and ready for use. In FIGS. 29 and 30, the lancing element 220 has been “fired” by the actuating mechanism such that the lancing element is in a second extended position where the needle point 226 extends beyond the endcap 214 by a second distance 230. With the endcap projection in a deeper recess, there is less distance between the housing body 236 and the user's skin, thereby increasing the depth of penetration. Accordingly, in this second extended position of the lancing element 220, the needle point 226 penetrates the skin by about the second distance 230 to form a wound for obtaining a sample of blood or other body fluid, with the wound being deeper than that produced with the projection in a shallower recess. In this way, a user selectively positions the endcap 214 relative to the housing 212, with a projection engaged in a selected recess (or disengaged from all recesses and against a forward end portion of the housing). By doing so, the distance to which the needle point 226 of the lancing element 220 extends beyond the forward face of the endcap 214 is varied, thereby adjusting the depth of penetration of the needle into the skin of the sampling site.

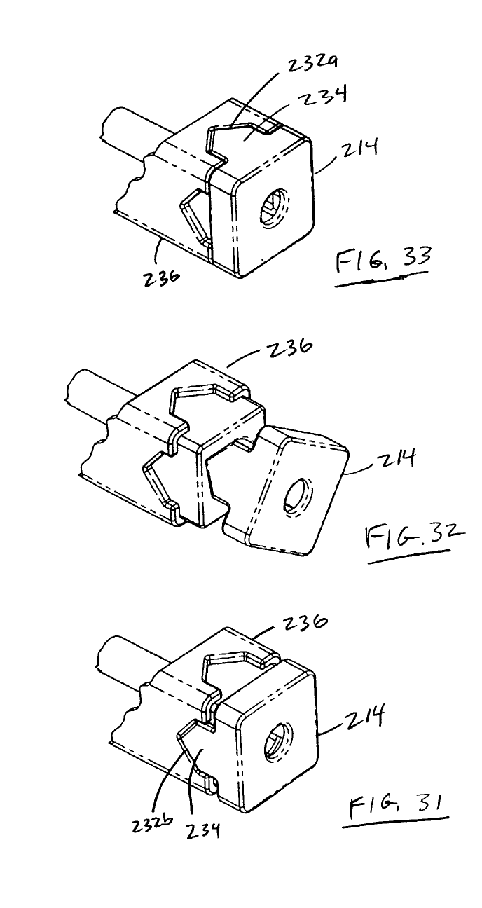

FIGS. 31-33 show the adjusting of the endcap 214. In FIG. 31, the endcap 214 is in the first position with the projection 234 in the deeper recess 232a. In FIG. 32, the endcap 214 is pulled away from the housing body 236 and rotated toward the desired projection/recess alignment. And in FIG. 33, the endcap 214 is repositioned into the second position with the projection 234 in the shallower recess 232b, thereby positioning the endcap further away from the body 236 in a spaced apart relationship.

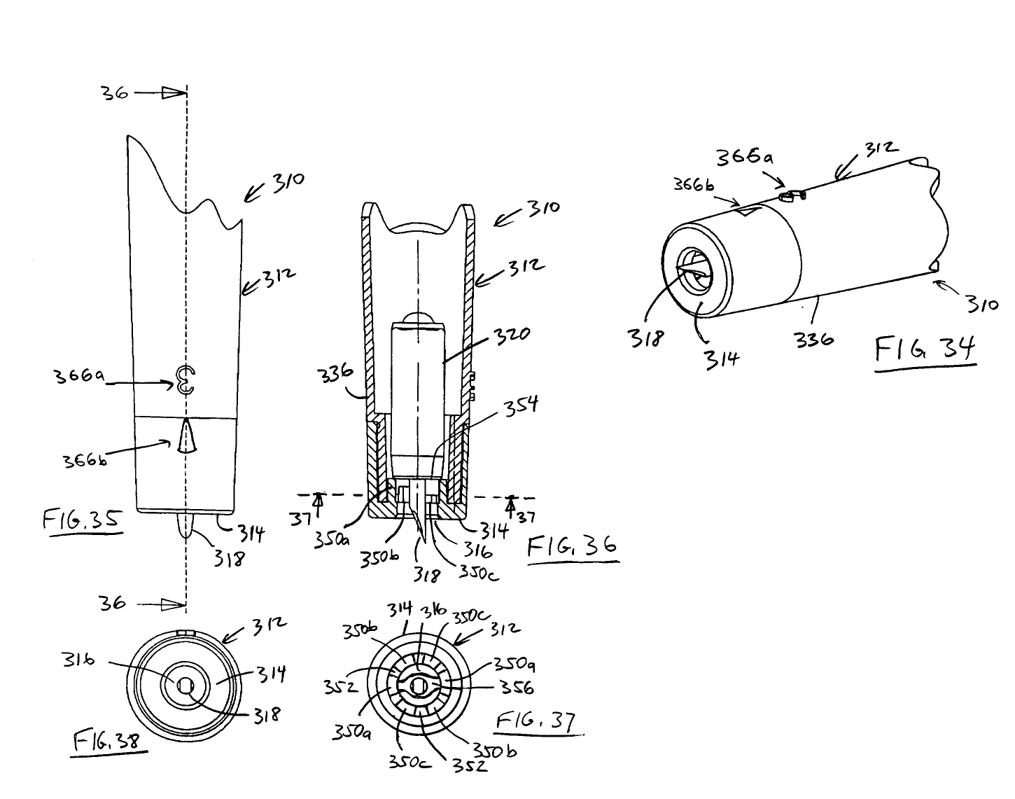

Referring to FIGS. 34-38, there is shown a third exemplary embodiment of the present invention, referred to generally as the lancing device 310. The lancing device 310 includes a housing 312 and an endcap 314 with an opening 316 for permitting at least a portion of a needle 318 of a lancet or other lancing element 320 to pass therethrough to penetrate the skin of a user for sampling. In this embodiment, the adjustment mechanism includes the endcap 314 being rotationally mounted to the housing body 336 by conventional means. One or more internal stops are provided, such as stops 350a, 350b, and 350c (collectively referred to as “the stops 350”). The stops 350 extend from or are defined on (flush with or recessed into) an inside face 352 of the endcap 314 at angularly offset position(s) around the opening 316. Each of the stops 350 may be provided by a pair of stop arms disposed at opposing sides of the opening, as best shown in FIGS. 36 and 37. Each of the stops 350 have a different length, and a cooperating portion 354 of the lancing element 320 impacts one of the stops 350 to limit the lancing element's travel and thereby the needle's 318 depth of penetration into the skin of the sampling site. For example, the cooperating portion 354 of the lancing element may be provided with one or more extension members 356 that impact the stops 350, so that the endcap 314 may be rotated so that only the desired stops are aligned with and impacted by the selected extension members.

It will be understood that many alternative forms of this embodiment may be provided. For example, one or more inclined ramp stops with a linear or curved surface (unshown) can be provided around the opening in place of the stop arms to provide continuous depth adjustment within a predetermined range. Or one or more stops in a staircase-like configuration (unshown) can be provided around the opening arms to provide multiple discrete depth adjustments. Alternatively, the endcap can be eccentrically shaped and rotatable so that rotating it positions the desired stop into alignment with the cooperating portion. And, of course, another number of the stops may be provided.

FIGS. 39-42 show the operation of the lancing device 310. The lancing device is positioned with the forward face of the endcap 314 in contact with the user's skin (not shown) at the desired sampling site. In FIGS. 39 and 40, the lancing element 320 is cocked in the retracted position and ready for use. In FIGS. 41 and 42, the lancing element 320 has been “fired” by an actuating mechanism such that the lancing element travels to a first extended position where at least a portion of the needle 318 passes through the opening 316 and a point 326 of the needle extends beyond the endcap 314 by a first distance 328. In the first extended position of the lancing element 320, the needle point 326 penetrates the skin by about the first distance 328 to form a wound for obtaining a sample of blood or other body fluid. With the endcap 314 in the first position, the cooperating portion 356 of the lancing element 320 impacts the longest stop 350a and produces the shallowest incision.

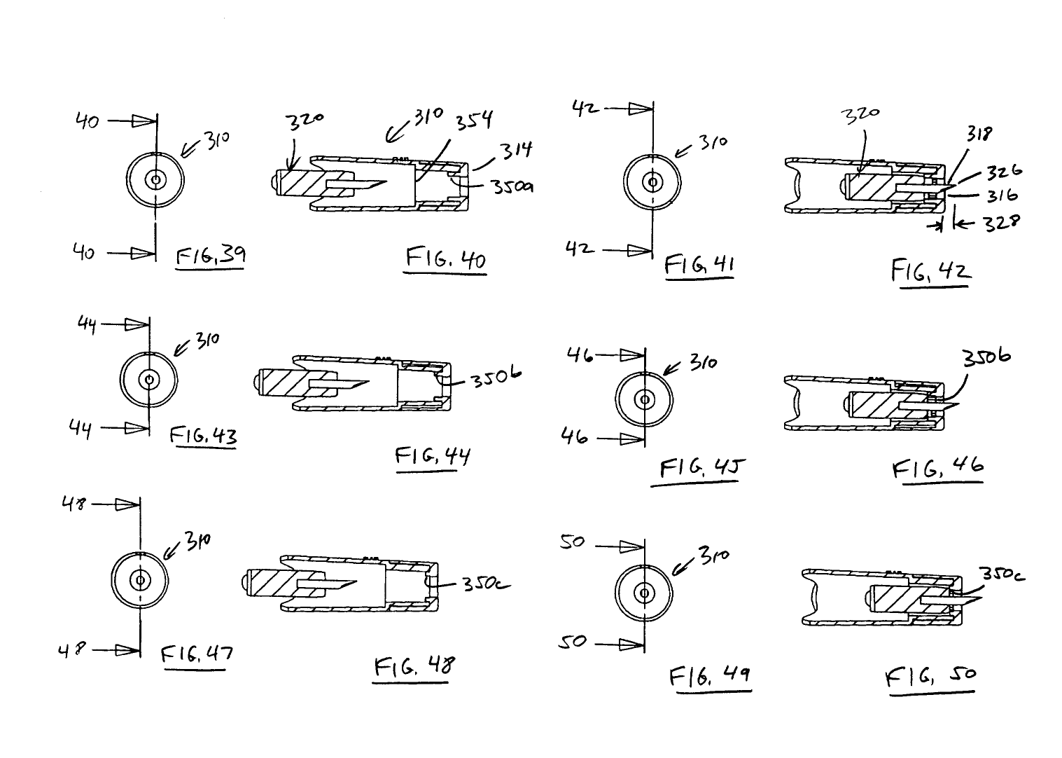

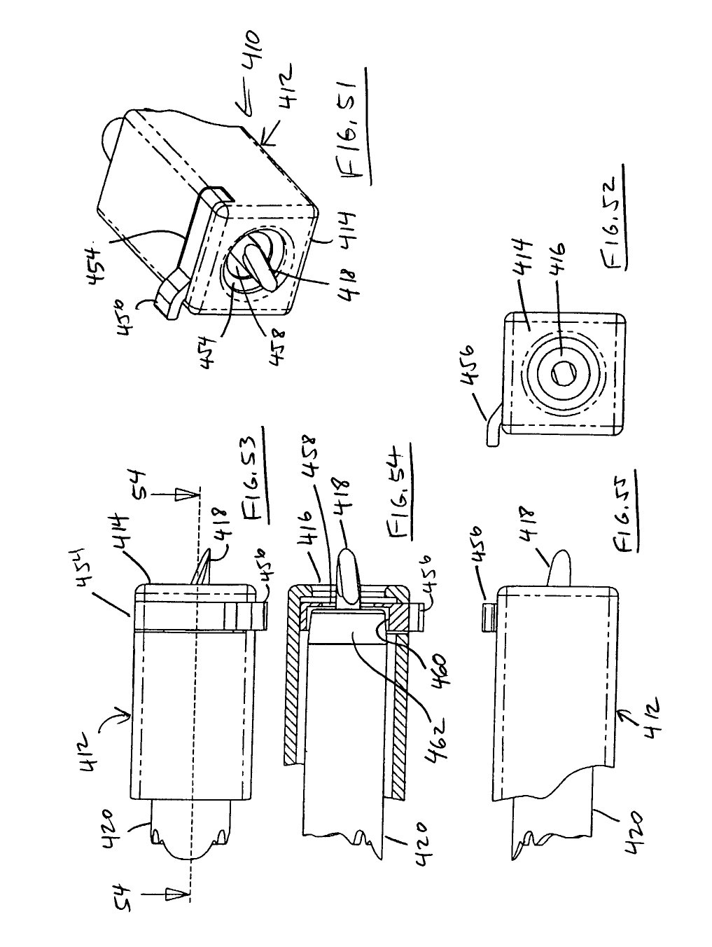

Similarly, FIGS. 43 through 50 show how the user can selectively vary the rotational position of the endcap 314 to determine which stop 350 is aligned for contact with the lancing element 320 to limit its travel. In this manner, the distance by which the point 326 of the needle 318 of the lancing element 320 projects through the opening 316, beyond the forward face of the endcap 314, and into the skin of the sampling site is adjusted. Impact of the lancing element 320 against the longer stop 350a allows less of the needle 318 to project beyond the front face of the endcap 314, and therefore will produce a shallower wound than impact with a shorter stop (as shown in FIGS. 39-42). Impact of the lancing element 320 against progressively shorter stops 350b, 350c, (including where the stop is defined on the inside face of the endcap) allows more and more of the length of the needle 318 to project beyond the front face of the endcap 314, and therefore will produce progressively deeper incisions (as shown in FIGS. 43-50).

Referring to FIGS. 51-55, there is shown a fourth exemplary embodiment of the present invention, referred to generally as the lancing device 410. The lancing device 410 includes a housing 412 and an endcap 414 with an opening 416 for permitting at least a portion of a needle 418 of a lancet or other lancing element 420 to pass therethrough to penetrate the skin of a user for sampling. In this embodiment, the adjustment mechanism includes a removable spacer 454 provided in the housing 412 adjacent the endcap 414. The spacer 454 preferably has a tab 456 to permit the user to more easily flip out the spacer for removal. The spacer 454 preferably has an opening 458 configured to align with the opening 416 through the endcap 414 when the spacer is in an inserted position within the lancing device 410. The openings 458, 416 are preferably sized and/or shaped to permit passage of a portion of a lancet or other lancing element 420, but block the entire lancing element from passing therethrough. In addition, the spacer 454 has a recess 460 formed in it for receiving the cooperating portion 462 of the lancing element 420 to permit the cooperating portion to travel to close to the end cap, thereby producing a small differential in the depths of penetrations provided (see FIG. 54). The spacer 454 is preferably pivotally mounted to the housing 412 and/or the endcap 414, as by a hinge or pivot pin, and moves between an inserted position within the lancing device 410 and a removed position at least partially outside the remainder of the lancing device.

It will be understood that many alternative forms of this embodiment may be provided. For example, while the housing and endcap are shown as rectangular, they could be circular, triangular, polygonal, or otherwise shaped. In addition, the spacer opening can be in the shape of a semi-circular slot so that when the spacer is pivoted partially out of the housing the lancing needle element can still pass through it, and the spacer recess can have different thicknesses (linear, curved, stepped, or otherwise varied). In this way, multiple penetration depths are provided depending on how far the spacer is pivoted out of the housing. And, of course, more than one spacer can be included, with a desired spacer pivoted into the housing to provide the desired penetration depth. For example, two spacers with different thickness recesses can alternately pivot into a single spacer bay in the housing from opposite sides of the housing, two spacers can be generally flat and pivot simultaneously into the same spacer bay from opposite sides or the same side of the housing, or two spacers can each have a dedicated spacer bay for being received into the housing.

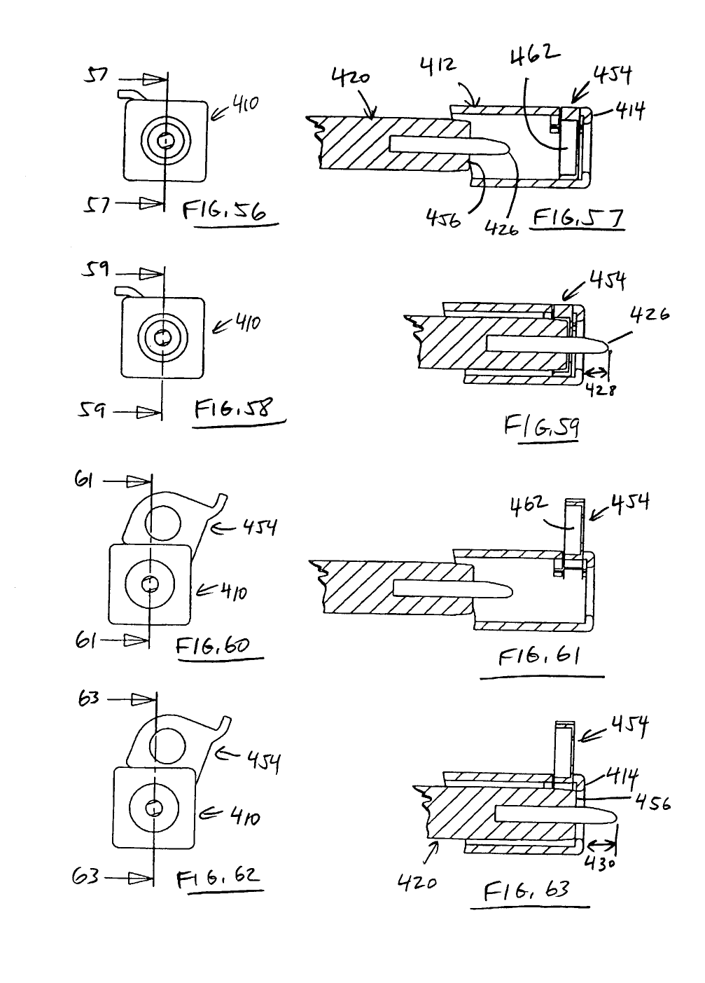

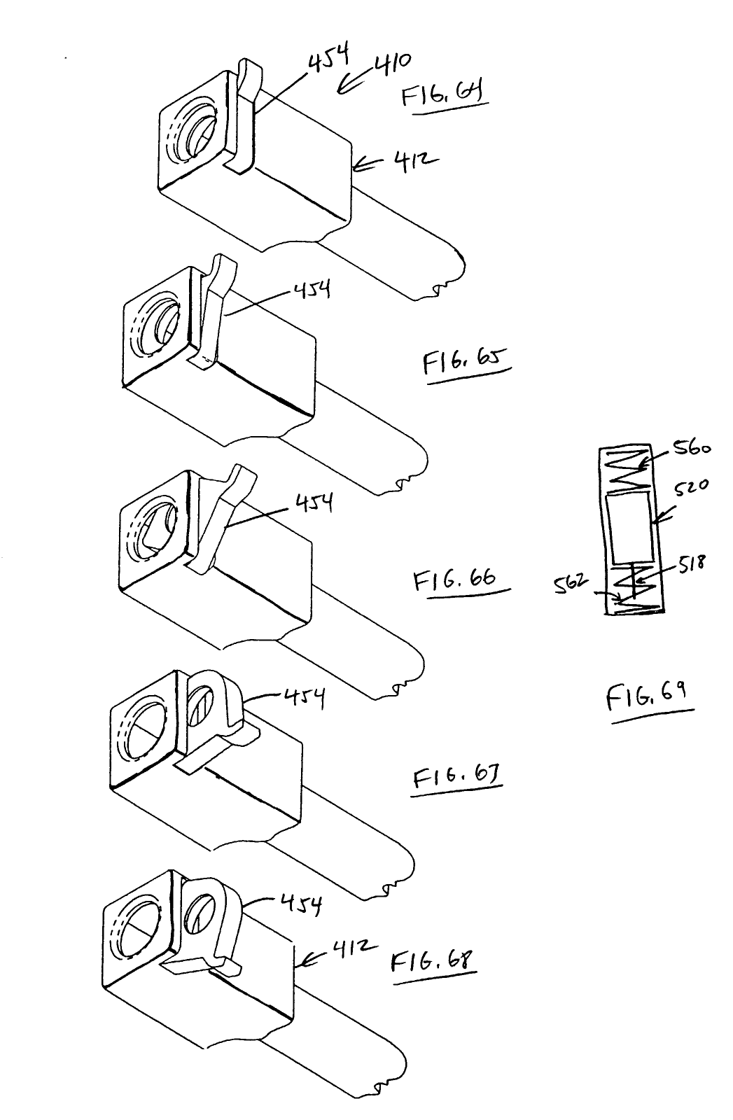

FIGS. 56-63 show the operation of the lancing device 410, including with the spacer 454 in the first position inserted into the housing 412 for providing a shallower lancing penetration (FIGS. 56-59) and with the spacer 454 in the second position pivoted out of the housing for providing a deeper lancing penetration (FIGS. 60-63). The lancing device 410 is positioned with the forward face of the endcap 414 in contact with the user's skin (not shown) at the desired sampling site. Similarly to the previously described embodiments, the lancing element 420 is “fired” by an actuating mechanism, from the retracted position (FIGS. 57 and 61) to the extended position (FIGS. 59 and 63). When the spacer 454 is in its inserted position (FIGS. 56-59), a forward face or other cooperating portion 456 of the lancing element 420 contacts the inner face of the spacer to limit travel of the lancing element. This limits the distance by which the needle point 426 extends beyond the forward end face of the endcap 414 to the first distance 428, and thereby reduces the depth of penetration into the sampling site. But when the spacer 454 is in its removed position (FIGS. 60-63), the lancing element 420 does not contact the spacer, and instead continues its travel in the extended direction until contacting the inner face of the endcap 414. This limits the travel of the lancing element 420 instead to the second distance 430, thereby increasing the distance by which the needle point 426 extends beyond the forward end face of the endcap 414 and increasing the depth of penetration into the sampling site.

FIGS. 64-68 show the adjusting of the spacer 454. In FIG. 64 the spacer 454 is in the first position inserted into the housing 412 so that it limits the travel of the lancing element. In FIGS. 65 through 67 the spacer 454 is progressively pivoted out of the housing. And in FIG. 68 the spacer 454 is removed from the housing 412 SO that it does not limit the travel of the lancing element.

Having described several exemplary embodiments of the penetration distance adjusting mechanism, additional details of other components of the lancing device will now be provided. In preferred form, the lancing device of the present invention, including for example any of the above-described embodiments, comprises a means for indicating whether the distance adjusting means is set for the needle to extend to the first distance from the housing, to the second distance, or to another distance. The indicating means may include cooperating positional indicia marked on, formed into, or otherwise applied to the housing body and/or the housing endcap for indicating the relative rotational position of the endcap to the housing body (and correspondingly the resultant depth of penetration). The positional indicia may include numerals, letters, alphanumeric designations, other symbols, pointers, and other indicia for designating the differences in depth penetration. For example, in the second embodiments of FIGS. 19 and 19A, positional indicia 266 and 266a are shown cooperating with the projection 234 and 234a to indicate the penetration depth setting. And in the third embodiment of FIGS. 34 and 35, positional indicia 366a are shown cooperating with positional indicia 366b to indicate the penetration depth setting.

Additionally, in preferred form the lancing device of the present invention, including for example any of the above-described embodiments, is a single-use lancing device, shown schematically in FIG. 69. The lancing device preferably includes the lancet or other lancing element 520 with the needle 518. The needle 518 may be provided by any component with a sharp edge or point capable of penetrating skin of the intended sampling site. A spring or other biasing means 560 is preferably provided for driving the lancing element 520 from the retracted position to the extended position to cause the point of the lancing element to penetrate the skin of the sampling site. A cocking mechanism (not shown) may be provided for loading the spring or other biasing means 560 to arm the lancing device, and a trigger mechanism (not shown) may be provided for releasing the biasing means 560 to drive the lancing element 520 toward its extended position.

The single-use lancing device preferably also comprises a disabling mechanism for preventing re-use of the device after a single use. For example, the disabling mechanism may comprise a return spring 562 for returning and retaining the sharp point of the lancet 520 back into the housing after a single use. Alternatively, the disabling mechanism may comprise break-away elements or a frangible link in the cocking, firing (spring), or triggering mechanism to prevent rearming or re-firing the device after a single use, a locking element, a shield for blocking travel of the lancet, and/or other means for disabling the lancing element including any that are known in the art. In addition, the single-use lancing device preferably includes a safety cap and sterile wrapping.

Alternatively, the lancing device of the present invention, including for example any of the above-described embodiments, is a reusable lancing device, not including the disabling mechanism, and preferably including means for rearming the device, removing and replacing the lancing element, and re-using the device for multiple samplings. These features are well known in the art. In further alternate embodiments, the lancing device of the present invention permits only a predetermined number of re-uses, and then disables the device from further re-use.

In still another alternative embodiment, the lancing device of the present invention is a single-use adjustable lancing attachment for use with a multiple-use firing mechanism. The firing mechanism is contained in a separate housing and may include a separate or integral cocking mechanism. In this way, the lancing element and the depth adjustment mechanism of the disposable attachment can be disposed of after a single use, while the firing mechanism is retained for future use with additional disposable attachments. Accordingly, this form includes a disconnect fitting for operationally coupling the disposable attachment to the firing mechanism, and the housing is typically shorter than in the embodiments described above. Otherwise, the adjustable lancing attachment is provided similarly to, for example, any of the above-described embodiments.

In the construction of any of the above-described embodiments, the lancing device is preferably fabricated primarily from plastics, as by injection molding. Alternatively, the lancing device may be formed from metals, ceramics, composites, or other materials.

In addition, the present invention includes a method of lancing the skin of a subject. In an exemplary method according to the invention, the steps include determining a desired lancing penetration depth, adjusting a housing of a lancing device to a setting for a lancing element that corresponds to the desired penetration depth, lancing the skin of the subject with the lancing device, and disabling and disposing of the lancing device after a single use to prevent further use after firing. The adjusting step may be accomplished by selectively removing a raised ring, selectively positioning an endcap of the lancing device to permit exposure of a desired length of the lancing element or to limit lancing element travel, or selectively pivoting a flip-out spacer of the lancing device to selectively limit lancing element travel. Details of these adjusting steps are provided above with respect to the various embodiments described herein.

Accordingly, the present invention provides a disposable, single-use lancing device with a penetration depth that can be adjusted, unlike all other known single-use lancets. Because the penetration depth can be adjusted, users can select the penetration depth produced by the lancing device to minimize pain and required healing time, while ensuring that the proper sample size can be collected from a sampling site using a single lancet stick. In addition, the penetration depth adjustment enables a user to obtain samples of the desired size from different sampling sites on the body. Furthermore, the depth adjustment feature is provided by structures that are sufficiently durable and reliable for use in reusable lancing devices.

While the invention has been disclosed in exemplary forms for illustration purposes, those skilled in the art will readily recognize that many modifications, additions, and deletions can be made therein without departing from the spirit and scope of the invention as set forth in the following claims.

What is claimed is:

1. A lancing device, comprising:
a housing having an endcap defining an opening; and
a lancing element having a needle with a point movable between a retracted position and one of at least two extended positions with the needle point extending through the opening, wherein the needle point extends a first distance from the endcap when in a first one of the extended positions and the needle point extends a second distance from the endcap when in a second one of the extended positions; and
a means for adjusting the distance that the needle point extends from the endcap between the first distance and the second distance.

2. The lancing device of claim 1, wherein the distance adjusting means comprises a raised ring surrounding at least a portion of the opening, wherein the raised ring is detachable from the remainder of the housing to permit exposing a greater length of the lancing element needle.

3. The lancing device of claim 1, wherein the distance adjusting means comprises one or more projections extending from the endcap for cooperative engagement with one or more recesses of the housing to permit repositioning the endcap to expose different lengths of the needle.

4. The lancing device of claim 1, wherein the distance adjusting means comprises at least two stops defined on or extending from an inside face of the endcap for selectively contacting a cooperating portion of the lancing element to limit lancing element travel.

5. The lancing device of claim 1, wherein the distance adjusting means comprises a flip-out spacer pivotally mounted adjacent an inside face of the endcap for contacting a cooperating portion of the lancing element in a first position of the flip-out spacer to limit lancing element travel and for avoiding contact with the lancing element in a second position of the flip-out spacer.

6. The lancing device of claim 1, further comprising a means for indicating whether the distance adjusting means is set for the needle to extend to the first distance from the housing or to the second distance.

7. The lancing device of claim 1, further comprising a disabling mechanism operable to retain the needle point within the housing after a single use of the lancing device for preventing its re-use.

8. A lancing device, comprising:
a housing having an endcap defining an opening and a raised ring surrounding at least a portion of the opening; and
a lancing element having a needle point movable between a retracted position and one of at least two extended positions with the needle point extending through the opening, wherein the needle point extends a first distance from the housing raised ring when in a first one of the extended positions and the needle point extends a second distance from the housing endcap when in a second one of the extended positions,
wherein the raised ring is detachable from the endcap, and the needle point extends the first distance from the housing raised ring when the ring is attached to the housing and the needle point extends the second distance from the housing endcap when the ring is removed from the housing.

9. The lancing device of claim 8, wherein the raised ring includes a pull tab for grasping to remove the ring from the housing.

10. The lancing device of claim 8 further comprising a disabling mechanism operable to retain the needle point within the housing after a single use of the lancing device for preventing its re-use.

11. A lancing device, comprising:
a housing having a body and an endcap defining an opening;
a lancing element having a needle point movable between a retracted position and one of at least two extended positions with the needle point extending through the opening, wherein the needle point extends a first distance from the endcap when in a first one of the extended positions and the needle point extends a second distance from the endcap when in a second one of the extended positions; and
one or more recesses that receive one or more projections for coupling the housing endcap to the housing body, the recesses and the projections defined in or extending from the housing body and the housing endcap to permit repositioning the endcap on the body to expose different lengths of the lancing element needle, wherein the needle point extends the first distance from the endcap when the endcap is coupled to the housing in a first position and the needle point extends the second distance from the endcap when the endcap is coupled to the housing in a second position.

12. The lancing device of claim 11, wherein the one or more recesses comprise at least two recesses with different lengths defined in the housing body and the one or more projections extend from the housing endcap so that the endcap is spaced apart from the body when in the second position.

13. The lancing device of claim 11, wherein the housing body has an inner wall with at least one catch surface defined thereon, the housing endcap has at least one guide member extending into the body, and the guide member has a retainer that engages the catch surface to prevent the endcap from being removed from the body.

14. The lancing device of claim 11, wherein the housing body has an inner wall with at least one stop surface defined thereon, and the lancing element has at least one stop member extending toward the inner wall, wherein the stop member contacts the stop surface when the lancing element is in the second position to limit lancing element travel.

15. The lancing device of claim 11, further comprising a means for indicating whether the endcap is positioned for the needle to extend to the first distance from the endcap or to the second distance.

16. The lancing device of claim 11, further comprising a disabling mechanism operable to retain the needle point within the housing after a single use of the lancing device for preventing its refuse.

17. A lancing device, comprising:
a housing having a body and an endcap rotationally coupled to the body and defining an opening;
a lancing element having a needle with a point movable between a retracted position and one of at least two extended positions with the needle point extending through the opening, wherein the needle point extends a first distance from the endcap when in a first one of the extended positions and the needle point extends a second distance from the endcap when in a second one of the extended positions; and
at least two stops defined on or extending from an inside face of the endcap for selectively contacting a cooperating portion of the lancing element to limit lancing element travel, wherein the needle point extends the first distance from the endcap when the endcap is rotated so that one of the stops contacts the lancing element and the needle point extends the second distance from the endcap when the endcap is rotated so that another one of the stops contacts the lancing element.

18. The lancing device of claim 17, wherein at least one of the stops comprises a pair of stop arms disposed at opposing sides of the opening.

19. The lancing device of claim 17, wherein at least one of the stops extends from the inside face of the endcap a greater distance than another one of the stops.

20. The lancing device of claim 17, further comprising a means for indicating whether the distance adjusting means is set for the needle to extend to the first distance from the housing or to the second distance.

21. The lancing device of claim 17, further comprising a disabling mechanism operable to retain the needle point within the housing after a single use of the lancing device for preventing its re-use.

22. A lancing device, comprising:
a housing having an endcap defining an opening;
a lancing element having a needle with a point movable between a retracted position and one of at least two extended positions with the needle point extending through the opening, wherein the needle point extends a first distance from the endcap when in a first one of the extended positions and the needle point extends a second distance from the endcap when in a second one of the extended positions; and
a flip-out spacer pivotally mounted adjacent an inside face of the endcap for contacting a cooperating portion of the lancing element in a first position of the flip-out spacer to limit lancing element travel and for avoiding contact with the lancing element in a second position of the flip-out spacer, wherein the needle point extends the first distance from the endcap when the flip-out spacer is in the first position and the needle point extends the second distance from the endcap when the flip-out spacer is in the second position.

23. The lancing device of claim 22, wherein the flip-out spacer defines a recess for receiving the cooperating portion of the lancing element to permit the cooperating portion to travel to close to the endcap.

24. The lancing device of claim 22, wherein the flip-out spacer includes a flip tab for grasping to pivot the spacer out of the housing to the second position of the flip-out spacer.

25. The lancing device of claim 22, further comprising a disabling mechanism operable to retain the needle point within the housing after a single use of the lancing device for preventing its re-use.

26. A method of lancing the skin of a subject, the method comprising:
determining a desired lancing penetration depth;
adjusting a housing of a lancing device to a setting for a lancing element that corresponds to the desired penetration depth;
lancing the skin of the subject with the lancing device; and
disabling further use of the lancing device after a single use.

27. The method of claim 26, wherein the adjusting step comprises selectively removing a raised ring of the lancing device to permit exposure of a desired length of the lancing element.

28. The method of claim 26, wherein the adjusting step comprises selectively positioning an endcap of the lancing device to permit exposure of a desired length of the lancing element or to limit lancing element travel.

29. The method of claim 28, wherein the adjusting step further comprises selectively positioning a projection of the endcap in a recess of the housing.

30. The method of claim 28, wherein the adjusting step further comprises selectively positioning an internal stop within the housing.

31. The method of claim 26, wherein the adjusting step comprises selectively pivoting a flip-out spacer of the lancing device to selectively limit lancing element travel.
